(19) United States
(12) Patent Application Publication
(10) Pub. No.: US 2017/0239658 A1
(43) Pub. Date: Aug. 24, 2017

(54) METHODS AND SYSTEMS FOR CONTROLLING THE SHEAR MODULUS OF GENOMIC LENGTH DSDNA MOLECULES

(71) Applicants: Ezra S. ABRAMS, Newton, MA (US); Christian T. BOLES, Bedford, MA (US); Yu CHEN, Princeton, NJ (US); James STURM, Priceton, NJ (US); Robert AUSTIN, Princeton, NJ (US); SAGE SCIENCE, INC., Beverly, MA (US); TRUSTEES OF PRINCETON UNIVERSITY, Princeton, NJ (US)

(72) Inventors: Ezra S. ABRAMS, Newton, MA (US); T. Christian BOLES, Bedford, MA (US); Yu CHEN, Princeton, NJ (US); James STURM, Princeton, NJ (US); Robert AUSTIN, Princeton, NJ (US)

(21) Appl. No.: 15/519,515

(22) PCT Filed: Oct. 16, 2015
(86) PCT No.: PCT/US15/56104

§ 371 Date: Apr. 14, 2017

Related U.S. Application Data
(60) Provisional application No. 62/064,890, filed on Oct. 16, 2014.

(51) Int. Cl. B01L 3/00; G01N 27/447; G01N 15/14; C12Q 1/68

(52) U.S. Cl. CPC B01L 3/502761; C12Q 1/6806; G01N 27/44791; G01N 15/1484; B01L 2200/0663; B01L 2400/0421; B01L 2400/0487; B01L 2200/0652; G01N 2015/149; G01N 2015/1493

(57) ABSTRACT

In some embodiments, a method for manipulating DNA molecules for use in a microfluidic device is provided, where the method may comprise providing a solution of a plurality of DNA molecules having a first radius of gyration under under a zero flow velocity, and maintaining the DNA molecules in a spherical shape under a flow velocity.

### CROSS-REFERENCE TO RELATED APPLICATIONS

This application claims priority to U.S. Provisional Patent Application No. 62/064,890, filed Oct. 16, 2014, and entitled “Methods and Systems for Controlling the Shear Modulus of Genomic Length dsDNA Molecules,” the disclosure of which is incorporated herein by reference in its entirety.

This invention was made with government support under Small Business Innovation Research (SBIR) Grant No. 143HG006818-01 awarded by the National Institutes of Health. The government has certain rights in the invention.

### BACKGROUND

The relative statistical length scale of double-stranded DNA molecules (dsDNA, the terms “DNA” and “dsDNA” are used interchangeably throughout the present disclosure) is set by the number of statistical segments N=L/κ in the polymer, where L is the total length of the polymer and κ is the Kuhn length, described below. If N>>1, the dsDNA is said to be very long.

There are generally three ways to sort extremely long dsDNA as a function of L: gel electrophoresis at very low fields to avoid elongation of the spherical random-coil polymer [6] (which requires multi-day run times), by full elongation in crossed fields [1, 9], or by stretching in microfabricated structures [22]. While stretching of a dsDNA polymer in post arrays or in microstructures or nanochannels is attractive and popular [12], it lacks the scalable high throughput that continuous flow particle separation technologies can provide [18, 21], which are necessary for many preparative applications [3].

Techniques which do not stretch dsDNA rely on a conformation of the genomic dsDNA which is as close to spherical form as possible. Indeed, the first attempt to sort dsDNA in a microfabricated device [23] failed precisely because the dsDNA was so easily elongated in shear fields.

### SUMMARY OF SOME OF THE EMBODIMENTS

To that end, in embodiments of the present disclosure, methods and systems are presented to control the conformation of DNA to a non-elongated configuration so that it can be easily handled, e.g., sorted, concentrated, and the like, using, for example, a deterministic lateral displacement array for concentrating DNA).

Accordingly, in some embodiments, a method for manipulating DNA molecules for use in a microfluidic device are provided. In such embodiments, the method comprises providing a solution of a plurality of DNA molecules which have an approximately spherical shape and a first radius of gyration under under a zero flow velocity. The DNA molecules may be maintained in an approximately spherical shape under a useful range of flow velocity in a microstructured environment. For some embodiments, a useful range of flow velocity is from about 1 micron/second to about 1 millimeter/second.

In some embodiments (similar to that noted above), a method for manipulating DNA molecules in a microfluidic device is provided, where the method comprises providing a solution of a plurality of DNA molecules having a first radius of gyration under under a zero flow velocity. The first radius of gyration of the DNA molecules may be maintained and/or decreased under a useful range of flow velocity.

In some embodiments, a method for manipulating DNA molecules in a microfluidic device is provided, where the method comprises providing a plurality of DNA molecules in a solution comprising agents that increase the shear modulus of the DNA molecules. These embodiments may be configured such that the DNA molecules are able to maintain an approximately spherical conformation under a useful range of flow velocity.

In some embodiments, methods may be provided for maintaining an approximately spherical shape of the DNA molecules, or maintaining and/or decreasing the first radius of gyration of the DNA molecules, or increasing the shear modulus of the DNA molecules. Each of these results may be achieved by adding an amount of DNA condensation agent to the solution. The DNA condensation agent may be selected from the group consisting of: polyethylene glycol polymers (PEG), polyvinylpyrrolidone, spermine, spermidine, cobalt hexamine, and cetyltrimethylammonium bromide (CTAB).

In some embodiments, the amount of DNA condensation agent is between:
about 1% to about 40% wt/vol.;
about 1% to about 20% wt/vol.;
about 5% to about 20% wt/vol.; or
about 10% to about 20% wt/vol.

In some embodiments, one and/or another of the disclosed methods include flowing the solution (i.e., DNA combined with the the DNA condensation agent) through a microfludic device, where the microfluidic device comprises at least one of: a deterministic lateral displacement (DLD) array, a Brownian rachet array, a pinched flow fractionation device, a hydrodynamic filtration device, and a anisotropic nanofilter array. In some embodiments, the microfluidic device manipulates the DNA molecules, where manipulation may comprise at least one of: fractionation by size, purification, chemical modification, and enzymatic modification.

In some embodiments, a system for concentrating DNA in a microfluidic environment is provided, and may comprise a deterministic lateral displacement (DLD) array. In some embodiments, the DLD array may comprise one or the other of DLD arrays described in references [5, 10, 11, 14, 15, 16], each of which are hereby incorported by reference. In some embodiments, the DLD array is configured to cause concentration of DNA molecules having a radius of gyration greater than a first (e.g., critical) radius from a solution of DNA molecules comprising an amount of DNA condensation agent. The system may also additionally comprise a source of hydrodynamic pressure configured to cause the solution to flow in the microfluidic channels at a fluid flow velocity. One and/or ther other of the fluid flow velocity and amount of DNA condensation agent are configured so as to cause the DNA molecules to remain in an approximately spherical conformation.

In some embodiments, a system for concentrating DNA in a microfluidic environment is provided, and may comprise a deterministic lateral displacement (DLD) array. The DLD array may be configured to cause concentration of DNA molecules having a radius of gyration greater than a first (e.g., critical) radius from a solution of DNA molecules comprising an amount of DNA condensation agent. The embodiment may use electric fields to move DNA molecules through the DLD arrays at an electrophoretic mobility. Electrophoretic fields may be continuous [for some embodiments, see, e.g., 26], or alternating in direction and duration as in pulsed field electrophoresis [for some ebodiments, see, e.g., 9]. The electrophoretic mobility and amount of DNA condensation agent are configured so as to cause the DNA molecules to remain in an approximately spherical conformation.

In some embodiments, a system for concentrating DNA in a microfluidic environment is provided, and may comprise a deterministic lateral displacement (DLD) array having a critical diameter for bumping within the range of about 0.2 to about 3 microns (for example).

In some embodiments, the fluid flow velocity ranges from about 1 μm/s to about 1000 μm/s.

As noted above, in some embodiments, a DNA condensation agent is added to a solution of DNA molecules, which may be (e.g.) polyethylene glycol polymers (PEG). The presence of PEG molecules reduces the intrinsic radius of gyration of the DNA molecules, resulting in segments of DNA overlaping at an intersection to reduce the total volume of depletion zones (an excluded volume between DNA and PEG), which the PEG molecule cannot occupy due to the exclusion interaction between DNA and PEG molecule. This results in an increase of the shear modulus of DNA in microfluidic structures (e.g., in some embodiments, the increase may be up to about 10⁴).

Using such methodology to control DNA conformation, DNA can then be flowed through a microfluidic device for certain purposes (such as sorting and/or concentrating). For example, the microfluidic device may comprise a deterministic lateral displacement (DLD) array, which enables the DNA to be concentrated and isolated.

### BRIEF DESCRIPTION OF THE DRAWINGS

FIG. 1 shows an example of depletion force induced by PEG molecule crowding according to some embodiments.

FIG. 2a shows a DNA concentrator schematic using a DLD array according to one of the embodiments.

FIG. 2b shows a composite micrograph of phage T4 DNA (166 kb) traveling through the microfluidic arrays according to one of the embodiments.

FIG. 2c shows a fluorescence intensity profile across the microfluidic array output for the embodiment shown in FIG. 2a-b.

FIG. 3a shows a fluorescent micrograph at zero flow velocity according to the embodiment shown in FIG. 2.

FIG. 3b shows a fluorescent micrograph at a 20 μm/s flow velocity according to the embodiment shown in FIG. 2.

FIG. 3c shows a fluorescent micrograph at a 20 μm/s flow velocity and with a 5% PEG volume fraction according to the embodiment shown in FIG. 2.

FIG. 3d shows a fluorescent micrograph at a 20 μm/s flow velocity and with a 10% PEG volume fraction according to the embodiment shown in FIG. 2.

FIG. 4a shows a chart of elongated and spherical DNA molecules as a function of flow velocity, shear stress, and PEG volume fraction.

FIG. 4b shows a chart of elongated and spherical DNA molecules as a function of shear stress and shear modulus.

FIG. 5 shows a chart of data marking DNA molecules that are bumped or zig-zag as a function of PEG volume fraction and flow velocity.

### DETAILED DESCRIPTION OF SOME OF THE EMBODIMENTS

Polymer dynamics studies begin by assuming a simple random walk in three-dimensional space with Kuhn step size κ equal to twice the persistence length Lₚ, and also include with respect to embodiments of the present disclosure (e.g., for statistical analysis) two physical constraints: (1) the (sequence dependent [8]) molecular Young's modulus Yᵢ contribution to the persistence length; and (2) the influence of self-avoidance which must be included in the statistical analysis. These physical constraints make the problem of truly understanding the dynamics of the polymer under shear a formidable problem.

Accordingly, the connection between the Lₚ and the intrinsic (molecular) Young's modulus Yᵢ is given by:

$${L}_{p} = \frac{{Y}_{i} {I}_{A}}{{k}_{B} T} \qquad ( 1 )$$

where I_{A} is the surface moment of inertia of the polymer. That is, the intrinsic bending rigidity Yᵢ of the polymer is folded into the thermodynamic parameter κ=2Lₚ which is not changed in the process of dsDNA compaction to first order in the distortion of the dsDNA molecular structure for the deformations discussed here [24].

In some embodiments, self-avoidance appears as a modification of the simple expected dependence L^{1/2} of the radius of gyration R_{g} of the dsDNA, where L is the total length of the polymer. Self-avoidance can be viewed in a mean-field approach as due to a repulsive interaction between segments of length κ caused by the interaction of irreducible excluded volume vₑₓ of each segment. In a mean field approximation [20] the local density of links ρ in a space of dimension d for a polymer of radius of gyration R_{g} is:

$$ρ \approx \frac{N}{{R}_{g}^{d}} \qquad ( 2 )$$

Since the excluded volume interaction energy Eₑₓ≈k_{B}TvₑₓNρ and the entropic energy due to expansion Eₑₙ≈k_{B}TR_{g}²/N_{κ}², the total free energy G_{f}=Eₑₓ+Eₑₙ is:

$${G}_{f} \approx {k}_{B} T ( {v}_{ex} \frac{{N}^{2}}{{R}_{g}^{d}} + \frac{{R}_{g}^{2}}{N {κ}^{2}} ) \qquad ( 3 )$$

Minimization of Eq. 3 with respect to R_{g} then yields that within the confines of Flory theory for self-avoidance in d=3 dimensions the radius of gyration (R_{g}) is:

$${R}_{g} \approx {v}_{ex}^{\frac{1}{5}} {N}^{\frac{3}{5}} {κ}^{\frac{2}{5}} \qquad ( 4 )$$

As discussed below, a 166 kbp T4 dsDNA molecule of length L≈56 μm includes a persistence length Lₚ of about 50 nm [24], and the measured average radius of gyration R_{g} (also discussed below) is about 1.4 μm. From Eq. 4, this yields vₑₓ≈3.1×10³ nm³.

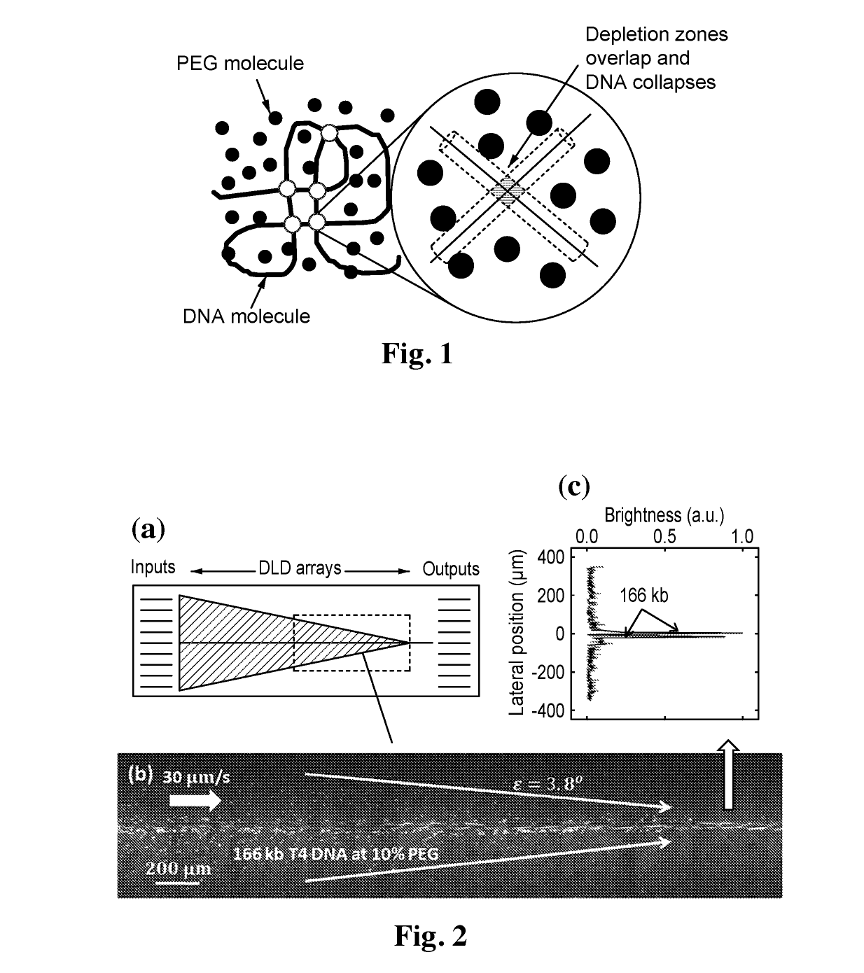

Depletion forces occur when the DNA solution also contains solutes that are much larger than the solvent molecules. To that end, in some embodiments, a small flexible polymer (in some embodiments, PEG) is added to a solution containing dsDNA in order to reduce the radius of gyration of the DNA molecule. The radius of gyration is a measure for the characterization of the time-averaged configuration of a polymer, which measures the root-mean-square distance of the collection of segments from the common center of mass of the segments. In such embodiments, the volume around each DNA molecule includes additional regions of excluded volume where the DNA molecule and PEG molecules are in close proximity, which may be referred to as a depletion zones. Other PEG molecules cannot occupy these zones due to the exclusion interaction between DNA and the closely apposed PEG molecules. The overlap of two segments of DNA (at an intersection) reduces the total volume of the depletion zones around the DNA molecule, thereby increasing the volume accessible to PEG molecule (see FIG. 1). This results in a network of smaller dsDNA chains, and also increases the entropy of the entire DNA+PEG system. In some embodiments, this may produce an unexpected result, as the reduction of the intrinsic radius of gyration of the DNA molecule caused by DNA condensation agents is accompanied by enhanced resistance of the DNA molecule to shear deformation in flow. This enhanced resistance makes it possible for the DNA molecules to maintain a spherical conformation, which in turn allows manipulation of DNA molecules in a variety of rapid, scalable, continuous flow separation methods, which cannot be used in the absence of condensation agents, as outlined below.

Although the “radius of gyration” is a convenient term when defining the principles of some of the embodiments of the present disclosure, the “effective” size that the DNA exhibits in microfluidic devices is only approximated by the calculated radius of gyration. In some embodiments, the exact relationship between the size of the DNA and radius of gyration is estimated using the approximation that the DNA molecules are hard spherical particles. Nevertheless, as shown below, under some conditions the two values appear to scale together, and therefore, the concept of radius of gyration is useful for describing the principles of the invention.

FIG. 1 illustrates an example of depletion force induced by PEG molecule crowding.

There is a minimum radius of gyration R_{g} which can be reached by depletion forces, since the persistence length Lₚ is not changed by the compaction process caused by the depletion forces. The total excluded Vₑₓ volume is then:

Vₑₓ=N×vₑₓ (5)

It can be assumed if this volume represents the smallest possible volume that the polymer can have at temperature T even at very high concentrations of PEG, then a minimum radius of gyration R_{g,m} is:

$${R}_{g , m} \approx {[ \frac{3 {V}_{ex}}{4 π} ]}^{1 / 3} \qquad ( 6 )$$

From the value for vₑₓ from Eq. 4, the minimum radius of gyration R_{g,m} can be computed from Eq. 6. For the T4 dsDNA molecule, the expected R_{g,m}≈75 nm, a factor of approximately 20 from the aqueous value and about twice the radius of the T4 capsid head [17].

Considering the entropic elasticity of polymer chains, the collective shear modulus G_{c} of the dsDNA network is roughly estimated as [20]:

$${G}_{c} \approx \frac{{n}^{*} {k}_{B} T}{\frac{4 π}{3} {R}_{g}^{3}} \qquad ( 7 )$$

where n* (in the range of 1 to N) is the number of effective strands. As the PEG volume fraction increases, more overlap regions are generated (as well as the DNA strands in between these regions), which leads to an increase of n*.

At zero PEG concentration, no DNA network is formed, n*=1, which yields G_{c,o}≈3.6×10⁻⁴ Pa (confirmed for small displacement using optical tweezer [24]). In the case of maximum compaction, n*=N, G_{c,max}≈k_{B}T/vₑₓ is independent of L since it essentially represents an incompressible core of a compacted polymer, due to the physical constraints of self-avoidance and the elastic energy stored in the persistence length. For the T4 dsDNA molecules discussed below, the maximum value of the shear modulus G_{c} may be about 1.3×10³ Pa.

Some embodiments of the present disclosure which use depletion force to hold dsDNA molecules in a relatively non-deformable, spherical conformation enable the use of, for example, rapid, scalable continuous flow methods for DNA manipulation [18, 21]. According to some embodiments, a DNA concentrator is provided which utilizes one or more DLD arrays to concentrate genomic length dsDNA molecules.

In such DLD arrays, particles smaller than a critical size D_{c} follow a laminar flow direction, weaving through array in a “zigzag” trajectory, while particles larger than the critical size are displaced laterally in the array (i.e., by posts at each column), following a migration angle ε in a bumping trajectory [5, 10, 11, 16, 14, 15].

FIG. 2(a) shows the schematic of a DLD apparatus according to some embodiments of the disclosure. In some embodiments, such DLD arrays may have 6.3 μm diameter posts arranged on an 8 μm grid, with the angle between the (two) grid axes at about ε=3.8°, giving a critical size D_{c} of about 0.7 μm. Using such a DLD array, a 166 kbp T4 dsDNA at 10% PEG volume fraction may enter an input region of the array. This may be concentrated and collected along the center wall at a peak flow velocity v_{f,max} in the middle of a gap of 30 μm/s (FIGS. 2(b) and 2(c)). Such embodiments (using the dsDNA spherical conformation produced according to some embodiments) yield increased separation rates and resolution compared with the performance that can be achieved with gel electrophoresis.

The spherical DNA conformation (produced according to some embodiments) is not deformed upon meeting the posts; instead, the polymer is pushed into adjacent stream lines. Otherwise, if the dsDNA is elongated, it follows the laminar flow direction in a zigzag trajectory (since its short axis length is below the critical size). Deformation of the polymer from the desired spherical shape may occur when the shear strain γ≈1. Thus, in some useful embodiments, the shear stress τ should not exceed G_{c} in the device. Since the hydrodynamic shear stress

$$τ = μ \frac{{dv}_{f}}{dz}$$

(where μ is the viscosity of solvent which depends on the PEG volume fraction [7]), the transverse velocity gradients

$$\frac{{dv}_{f}}{dz}$$

that can be tolerated:

$$\frac{{dv}_{f}}{dz} max \approx \frac{{G}_{c}}{μ} \qquad ( 8 )$$

FIG. 2 illustrates the following:
FIG. 2a shows an embodiment of the DNA concentrator schematic using a DLD array;
FIG. 2b illustrates an embodiment with micrograph composite of purified 166 kbp T4 dsDNA in a peak velocity 30 μm/s flow at 10% PEG volume fraction traveling through the microfluidic arrays;
FIG. 2c illustrates the fluorescence intensity profile at the outputs indicates a good isolation of DNA molecules, according to some embodiments. The DNA was concentrated from a stream width of ˜600 micrometers near the device input (left side of FIG. 2) to less than 50 micrometers near the output.

EXAMPLE: DNA sample preparation (according to some embodiments). Phage T4 dsDNA (strain GT7, Wako) was equilibrated with YOYO1 (Life Technologies) at 50° C. for 1 hour under the following conditions: 10 mM Tris-HCl, pH 8.0, 1 mM EDTA, 0.5 M NaCl, 1 ng/L T4 dsDNA, 0.15 M YOYO1, 10 mM DTT (the ratio of YOYO1/DNA bp=1/10). Following equilibration, the YOYO1-labeled DNA was cooled to room temperature and diluted 100-fold with a buffer containing 10 mM Tris-HCl, pH 8.0, 1 mM EDTA, 10 mM DTT, and from 0 to 20% (w/v) PEG (Dow Carbowax Polyethylene Glycol 8000, average molecular weight 6000-9000) as indicated. The DNA solutions were stored at 4° C. in opaque plastic bottles until used. All solutions (except for those containing DNA) were filtered through 0.45 nm filters.

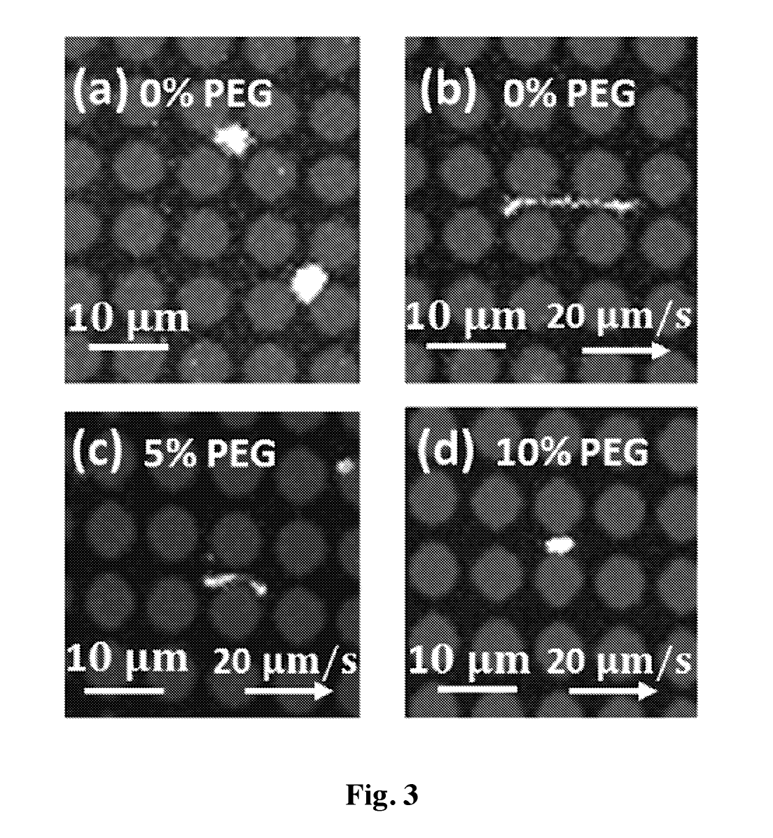

FIG. 3 shows fluorescent micrographs of purified 166 kbp T4 dsDNA under different conditions in the DLD arrays according to some embodiments of the present disclosure. In particular, FIGS. 3(a)-(d) show purified 166 kbp T4 dsDNA in microfluidic array at:
(a) zero flow velocity, where the DNA coils up into a sphere;
(b) 20 μm/s flow velocity with 0% PEG volume fraction where the DNA is elongated and stretched under the shear flow; p1 (c) 20 μm/s flow velocity with 5% PEG volume fraction, where DNA is less stretched in PEG solution under the shear flow; and
(d) 20 μm/s flow velocity with 10% PEG volume fraction, where DNA maintains the spherical conformation and behaves like a solid particle.
   Accordingly, at zero fluid velocity, DNA includes a spherical conformation, with an observed radius of 1.4 μm (FIG. 3(a)). When a fluid flow is inputted in the structure, with an average flow in a direction 3.8° from an array axis (Dc for the DLD array was approximately 0.7 μm), a velocity shear profile is then introduced as the fluid flows through the gaps (because of a boundary condition of zero velocity at the post walls). For a peak velocity v_{f,max} of 20 μm/s, DNA is observed with an elongated conformation, with a length as long as 20 μm, as shown in FIG. 3(b). However, in accordance with the solution of dsDNA and PEG according to some embodiments of the present disclosure, under fluid flow, the length of dsDNA is shorter, as shown in FIG. 3(c) under a 5% PEG volume fraction, and has about a zero elongation as shown in FIG. 3(d) at 10% PEG volume fraction (both at a peak velocity of about 20 μm/s). This is due to the increase of collective shear modulus G_{c}.

Further, the relationship between flow rate, PEG concentration, shear strain, and DNA conformation may be determined by analyzing video micrographs of T4 DNA molecules traversing DLD arrays and classifying the conformation of the DNA molecules according to the following convention. If the length is larger than about four times the radius of gyration R_{g} (≈6 μm, the shear strain γ>1), DNA molecules are classified as elongated; otherwise they are classified as spherical.

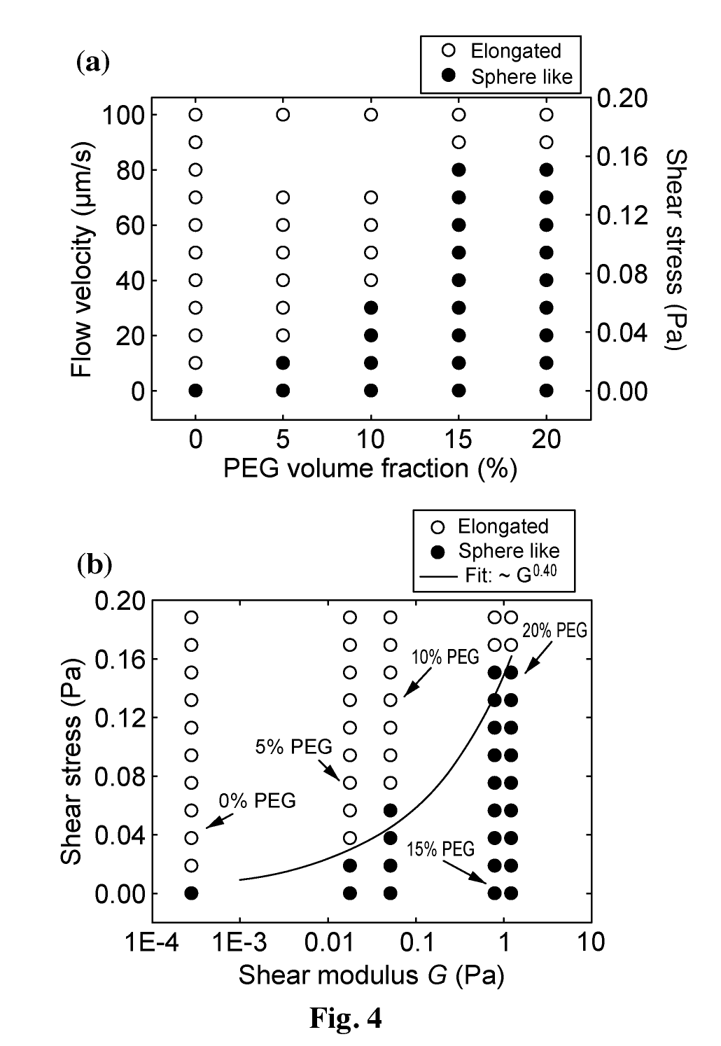

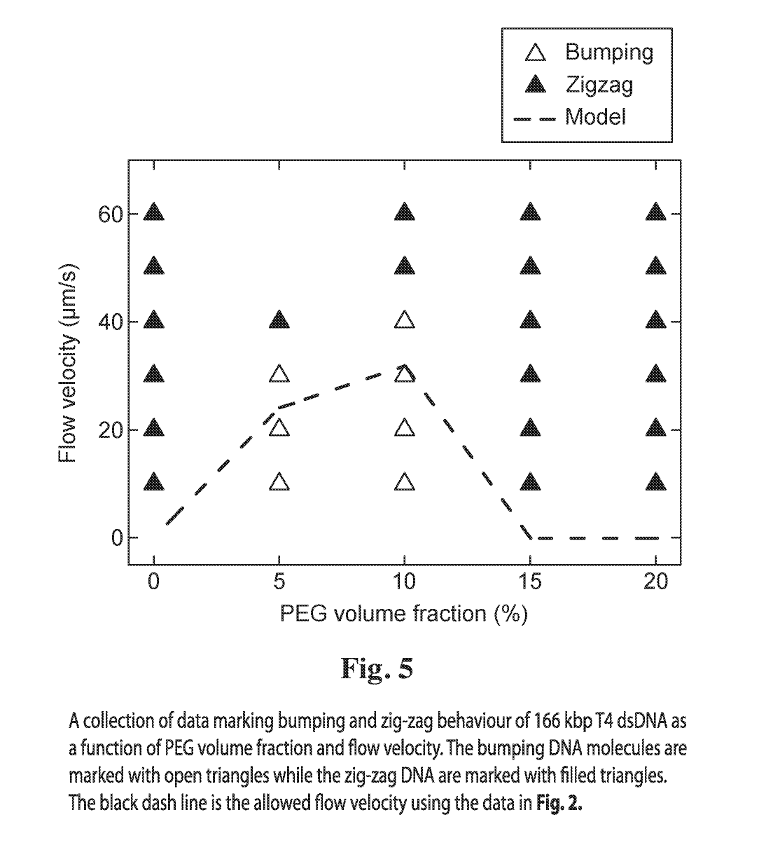

The results are shown in FIG. 4, where the open circles represent elongated DNA molecules and filled circles represent spherical DNA molecules. FIG. 4a illustrates a collection of data marking the “elongated” and “spherical” 166 kbp T4 dsDNA as a function of PEG volume fraction and flow velocity.

As shown in FIG. 4, the induced depletion force has an effect on the spherical-elongated phase transition. Further, introducing the depletion forces to the environment increases the shear modulus by a factor of about 10⁴. Thus, assuming a parabolic flow profile in the gap [11], the maximum transverse velocity gradients

$$\frac{{dv}_{f}}{dz}$$

in the microfluidic arrays can be written as:

$$\frac{{dv}_{f}}{dz} max = \frac{4 {v}_{f , max}}{d} \qquad ( 9 )$$

where d=1.7 μm is the minimum gap of this microfluidic structure. The collective shear modulus can be obtained from Eq. 8 and plotted in FIG. 4b with a black line. This means that the DNA's behavior in DLD arrays can be predicted (i.e., it follows the trajectory shown by the black dashed line). Below this line is the region of conditions for DNA flows in a bumping trajectory, while out of this region (i.e., above this line) DNA will follow a zig-zag trajectory.

A collection of data marking bumping and zig-zag 166 kbp T4 dsDNA as a function of PEG volume fraction and flow velocity is also shown in FIG. 5. Here, bumping DNA molecules are marked with open triangles, and zig-zag DNA molecules are marked with filled triangles. Accordingly, as the PEG volume fraction increases above 15%, DNA will undergo a coil-globule transition [19], and R_{g} will fall below the critical size D_{c}, leading to a sharp drop of the prediction at high PEG volume fraction regime. This results in DNA being too compacted to be displaced in the single DLD array used for the experiments shown in FIG. 5 (although it still has a spherical conformation). In some embodiments, a DLD array having a smaller critical size for bumping is provided, and thus, a DNA with a 20% PEG may also be bumped.

The black dash line shown on FIG. 5 represents the allowed flow velocity (using data from FIG. 2). At low PEG volume fractions, DNA can be collected at a high flow velocity, i.e., using a higher velocity flow than predicted. This may be because the short axis length of the DNA coil is still above the DLD array critical size even if DNA has apartially elongated conformation.

It should be pointed out that, in some embodiments, the effective size of the DNA for determining its behavior in the DLD array (that is, whether it bumps or not) may scale as the radius of gyration. Accordingly, for some embodiments, an important principle is that in the presence of condensing agents such as PEG, the effective size of the DNA as it moves through the DLD array doesn't decrease dramatically when the microfluidic flow velocity increases.

Thus, according to some embodiments of the present disclosure, dsDNA conformation can be controlled by molecular crowding induced depletion force via adjusting the shear modulus of DNA in microfluidic structures through the addition of PEG in determined amounts. This results in a simplified model and an increase in shear modulus of about 10⁴. In some embodiments, by controlling DNA conformation, DNA can be concentrated and isolated using DLD arrays in hydrodynamic fluid flow with higher separation rate and resolution than in current preparative electrophoresis methods.

Also, various inventive concepts may be embodied as one or more methods and systems of which examples have been provided herein. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Any and all references to publications or other documents, including but not limited to, patents, patent applications, articles, webpages, books, etc., presented anywhere in the present application, are herein incorporated by reference in their entirety.

Although a few variations have been described in detail above, other modifications are possible. For example, any logic flows depicted in the accompanying figures and/or described herein do not require the particular order shown, or sequential order, to achieve desirable results. Other implementations may be within the scope of at least some of the following exemplary claims.

As noted elsewhere, these embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, systems and apparatuses/devices which may further include any and all elements from any other disclosed methods, systems, and devices, including any and all elements corresponding to binding event determinative systems, devices and methods. In other words, elements from one or another disclosed embodiments may be interchangeable with elements from other disclosed embodiments. In addition, one or more features/elements of disclosed embodiments may be removed and still result in patentable subject matter (and thus, resulting in yet more embodiments of the subject disclosure). Also, some embodiments correspond to systems, devices and methods which specifically lack one and/or another element, structure, and/or steps (as applicable), as compared to teachings of the prior art, and therefore represent patentable subject matter and are distinguishable therefrom (i.e. claims directed to such embodiments may contain negative limitations to note the lack of one or more features prior art teachings).

All definitions, as defined and used herein, should be understood to control over dictionary definitions, definitions in documents incorporated by reference, and/or ordinary meanings of the defined terms.

### REFERENCES

[1] Olgica Bakajin, Thomas A. J. Duke, Jonas Tegenfeldt, Chia-Fu Chou, Shirley S Chan, Robert H Austin, and Edward C Cox. Separation of 100-kilobase DNA molecules in 10 seconds. Anal. Chem., 73(24):6053-6056, 2001.
[2] Thierry Biben, Peter Bladon, and Daan Frenkel. Depletion effects in binary hard-sphere fluids. J. Phys.: Condens. Matter, 8(50):10799, 1996.
[3] Erik Borgstrom, Sverker Lundin, and Joakim Lundeberg. Large scale library generation for high throughput sequencing. PLoS One, 6(4):e19119, 2011.
[4] Sónia Cunha, Conrad L Woldringh, and Theo Odijk. Polymer-mediated compaction and internal dynamics of isolated<i>escherichia coli</i> nucleoids. J. Struct. Biol., 136(1):53-66, 2001.
[5] John A Davis, David W Inglis, Keith J Morton, David A Lawrence, Lotien R Huang, Stephen Y Chou, James C Sturm, and Robert H Austin. Deterministic hydrodynamics: taking blood apart. Proc. Natl. Acad. Sci. U.S.A., 103(40):14779-14784, 2006.
[6] T. A. J. Duke. Monte carlo reptation model of gel electrophoresis: steady state behavior. J. Chem. Phys., 93(12):9049-9054, 1990.
[7] Latifah Binti Hamzah. The effect of viscoelastic fluids on flows generated by spherical objects during sedimentation. PhD thesis, Massachusetts Institute of Technology, 2012.
[8] M. E. Hogan and R. H. Austin. Importance of DNA stiffness in protein—DNA binding specificity. Nature, 329:263, 1987.
[9] Lotien Richard Huang, Jonas O Tegenfeldt, Jessica J Kraeft, James C Sturm, Robert H Austin, and Edward C Cox. A DNA prism for high-speed continuous fractionation of large DNA molecules. Nat. Biotechnol., 20(10):1048-1051, 2002.
[10] David W Inglis, John A Davis, Robert H Austin, and James C Sturm. Critical particle size for fractionation by deterministic lateral displacement. Lab Chip, 6(5):655-658, 2006.
[11] David W Inglis, John A Davis, Thomas J Zieziulewicz, David A Lawrence, Robert H Austin, and James C Sturm. Determining blood cell size using microfluidic hydrodynamics. J. Immunol. Methods, 329(1):151-156, 2008.
[12] Ernest T Lam, Alex Hastie, Chin Lin, Dean Ehrlich, Somes K Das, Michael D Austin, Paru Deshpande, Han Cao, Niranjan Nagarajan, Ming Xiao, et al. Genome mapping on nanochannel arrays for structural variation analysis and sequence assembly. Nat. Biotechnol., 30(8):771-776, 2012.
[13] L. S. Lerman. A transition to a compact form of DNA in polymer solutions. Proc. Nat. Acad. Sci. U.S.A., 68(8):1886-1890, 1971.
[14] Kevin Loutherback, Kevin S Chou, Jonathan Newman, Jason Puchalla, Robert H Austin, and James C Sturm. Improved performance of deterministic lateral displacement arrays with triangular posts. Microfluid. nanofluid., 9(6):1143-1149, 2010.
[15] Kevin Loutherback, Joseph D'Silva, Liyu Liu, Amy Wu, Robert H Austin, and James C Sturm. Deterministic separation of cancer cells from blood at 10 ml/min. AIP advances, 2(4), 2012.
[16] Kevin Loutherback, Jason Puchalla, Robert H Austin, and James C Sturm. Deterministic microfluidic ratchet. Phys. Rev. Lett., 102(4):045301, 2009.
[17] Norman H Olson, Mari Gingery, Frederick A Eiserling, and Timothy S Baker. The structure of isometric capsids of bacteriophage t4. Virology, 279(2):385-391, 2001.
[18] Nicole Pamme. Continuous flow separations in microfluidic devices. Lab Chip, 7(12):1644-1659, 2007.
[19] James Pelletier, Ken Halvorsen, Bae-Yeun Ha, Raffaella Paparcone, Steven J Sandler, Conrad L Woldringh, Wesley P Wong, and Suckjoon Jun. Physical manipulation of the escherichia coli chromosome reveals its soft nature. Proc. Natl. Acad. Sci. U.S.A., 109(40):E2649E2656, 2012.
[20] M Rubinstein and R. H. Colby. Polymer Physics. Oxford University Press, New York, 2003.
[21] Mark D. Tarn, Maria J. Lopez-Martinez, and Nicole Pamme. On-chip processing of particles and cells via multilaminar flow streams. Anal. Bioanal. Chem., 406:139-161, 2014.
[22] Jonas O Tegenfeldt, Christelle Prinz, Han Cao, Steven Chou, Walter W Reisner, Robert Riehn, Yan Mei Wang, Edward C Cox, James C Sturm, Pascal Silberzan, et al. The dynamics of genomic-length DNA molecules in 100-nm channels. Proc. Natl. Acad. Sci. U.S.A., 101(30):10979-10983, 2004.
[23] W. D. Volkmuth and R. H. Austin. DNA electrophoresis in microlithographic arrays. Nature, 358(6387):600-602, 1992.
[24] Michelle D Wang, Hong Yin, Robert Landick, Jeff Gelles, and Steven M Block. Stretching DNA with optical tweezers. Biophys. J., 72(3):1335-1346, 1997.
[25] Steven B Zimmerman and Allen P Minton. Macromolecular crowding: biochemical, biophysical, and physiological consequences. Annu. Rev. Biophys. Biomol. Struct., 22(1):27-65, 1993.
[26] Lotien Richard Huang, Edward C. Cox, Robert H. Austin, and James C. Sturm. Continuous Particle Separation Through Deterministic Lateral Displacement. Science, 304, 987-990, 2004.

1. A method for manipulating DNA molecules for use in a microfluidic device, the method comprising:
providing a solution of a plurality of DNA molecules having a set of first values for their radii of gyration, said first radii values varying in proportion to the molecular weight of each DNA molecule, under under a zero transport velocity; and
substantially maintaining the DNA molecules in a spherical shape under a transport velocity.

2. A method for manipulating DNA molecules in a microfluidic device, the method comprising:
providing a solution of a plurality of DNA molecules having a set of first values for their radii of gyration, said first radii values varying in proportion to the molecular weight of each DNA molecule, under under a zero transport velocity; and
providing conditions wherein the DNA molecules:
substantially maintain their first radii values under a transport velocity, and/or
decrease their radii of gyration to a set of second values for their radii of gyration, said second radii of gyration varying in proportion to the molecular weight of each DNA under a transport velocity.

3. The method of claim 1, wherein maintaining the spherical shape of the DNA molecules, or maintaining and/or decreasing the set of first values for their radii of gyration of the DNA molecules comprises adding an amount of DNA condensation agent to the solution.

4. The method of claim 1, wherein the DNA molecules are transported through the microfluidic device by flow.

5. The method of claim 1, wherein the DNA molecules are transported through the microfluidic device by electric fields.

6. The mcthon method of claim 1, wherein the DNA molecules are transported through the microfluidic device by a combination of flow and electric fields.

7. The method of claim 3, wherein the DNA condensation agent is selected from the group consisting of: polyethylene glycol polymers (PEG), Polyvinylpyrrolidone, Spermine, Spermidine, Cobalt hexamine, and Cetyltrimethylammonium bromide (CTAB).

8. The method of claim 3, wherein the amount of DNA condensation agent is between about 1% to about 40% wt/vol.

9. The method of claim 3, wherein the amount of DNA condensation agent is between about 1% to about 20% wt/vol.

10. The method of claim 3, wherein the amount of DNA condensation agent is between about 5% to about 20% wt/vol.

11. The method of claim 3, wherein the amount of DNA condensation agent is between about 10% to about 20% wt/vol.

12. The method of claim 1, further comprising flowing the solution through a microfludic device.

13. The method of claim 12, wherein the microfluidic device is selected from the group consisting of: a deterministic lateral displacement (DLD) array, a Brownian rachet array, a pinched flow fractionation device, a hydrodynamic filtration device, and a anisotropic nanofilter array.

14. The method of claim 12, wherein the microfluidic device manipulates the DNA molecules, wherein manipulation comprises at least one of: fractionation by size, purification, and chemical modification.

15. The method of claim 14, wherein chemical modification comprises enzymatic mofication.

16. A system for manipulating DNA in a microfluidic environment, comprising:
a deterministic lateral displacement (DLD) array, wherein the dimensions of the array are configured to fractionate DNA molecules on the basis of size, such that DNA molecules greater than a critical size move in a first direction through the array, and DNA molecules less than a critical size move in a second direction through the array;a DNA condensation agent; and
a transporter configured to cause the DNA molecules to move through in the microfluidic array at a transport velocity, wherein:
at least one of the transport velocity and amount of DNA condensation agent are configured such that the DNA molecules retain an approximately spherical conformation as they pass through the microfluidic array.

17. The system of claim 16, wherein the tranporter comprises a flow.

18. The system of claim 16, wherein the tranporter comprises at least one electric field.

19. The system of claim 16, wherein the transporter comprises a combinaion of a flow and at least one electric field.

20. The system of claim 16, wherein the DLD array is configured with a critical size for fractionation, expressed as a particle diameter of between 0.1 and 3 μm.

21. The system of claim 16, wherein the condensation agent causes the DNA molecules of the sample to adopt a more compact size when passing through the DLD array at the transport velocity.
